(19) United States
(12) Patent Application Publication
(10) Pub. No.: US 2021/0366614 A1
(43) Pub. Date: Nov. 25, 2021

(54) HAIR AND SCALP DIAGNOSIS AND TREATMENT

(71) Applicant: T-BIOMAX SDN BHD, Selangor (MY)

(72) Inventor: Thye CHEE CHONG, Subang Jaya (MY)

(73) Assignee: T-BioMax SDN BHD, Selangor (MY)

(21) Appl. No.: 16/980,351

(22) PCT Filed: Mar. 12, 2019
(86) PCT No.: PCT/MY2019/050019

§ 371 Date: Sep. 11, 2020

(30) Foreign Application Priority Data
Mar. 12, 2018 (MY) PI 2018700974
Mar. 13, 2018 (MY) PI 2018701003
Nov. 28, 2018 (MY) PI 2018002211

(51) Int. Cl. G16H 50/20; A61B 5/00; G16H 10/20; G16H 30/40; G06F 40/40; G16H 50/30

(52) U.S. Cl. CPC G16H 50/20; A61B 5/446; A61B 5/448; A61B 5/7264; A61B 5/7475; A61B 5/7246; A61B 2560/02; A61B 5/4836; G16H 10/20; G16H 30/40; G06F 40/40; G16H 50/30; A61B 5/0022

(57) ABSTRACT

The present invention provides a system and method for diagnosis and treatment of hair and scalp. The system comprises an electronic user interface, a plurality of data blocks configured to collect hair and scalp related data from a plurality of IOT devices, a processor coupled to an AI engine configured to process the data for identifying a problem and recommending a treatment, and a controller encoded with instructions to process a plurality of data models wherein the AI engine determines identifiers in the data based on at least one of the plurality of data models for analyzing condition of hair and scalp to recommend the treatment to a user on the interface.

### FIELD OF THE INVENTION

The present invention generally relates to diagnosis system and methods. More particularly, the invention relates to system and method for diagnosis and treatment of hair and scalp of a patient.

### BACKGROUND

Scalp and hair treatment are greatly in demand across age groups and across different countries. These days changing nature of jobs, work stress, sedentary lifestyle, increasing environmental pollution, extreme weather events, seasonal changes, hormonal fluctuation, and other variables affect scalp conditions and result in hair problems, such as hair fall.

Conventional methods for hair care include collecting customer information and providing appropriate products and services. However, conventional methods suffer from many challenges. First, there are a number of products and services offered by competing providers (e.g., cosmetic manufacturers, spas, salons, department stores, etc.). Hence, the customer becomes overwhelmed by his/her array of possible choices between all of the services and products available in the marketplace. Further, some of the conventional methods rely on questionnaires to make a determination of a patient's perceptions of their skin type, condition and needs. Such methods suffer from quality of services and hair care products given to the customer.

Further, some of the conventional methods provide scanning customer hair and scalp and provides hair care products by a professional based on the scanning information. However, due to the process followed by the existing scalp scanner the scanning reports are not efficient and takes more time. Further, the existing systems require professional assistance in understanding the reports generated, determining scalp type, potential factors contributing to hair conditions, understanding the required treatments to be carried out and the product to be used. The resulting information allows the customer to choose the most appropriate hair care products in order to maintain or improve hair conditions.

These conventional methods for the hair and scalp diagnosis solve some of the challenges, but still suffer from other issues as stated above. For example, they rely on professional support in understanding the report, understanding the kind of products that are suitable to their scalp and hair type, picking the right product among a gamut of choices available. In view of the forgoing discussion, there is a need for an improved system and method for diagnosing hair and scalp condition and recommending a suitable treatment and product thereof.

### SUMMARY OF THE INVENTION

Accordingly, the present invention provides an Artificial intelligence-based hair and scalp diagnosis system. The system comprises an electronic user interface, a plurality of data blocks configured to collect hair and scalp related data from a plurality of IOT devices, a processor coupled to an AI engine configured to process the data for identifying a problem and recommending a treatment, and a controller encoded with instructions to process a plurality of data models wherein the AI engine determines identifiers in the data based on at least one of the plurality of data models for analyzing condition of hair and scalp to recommend the treatment to a user on the interface.

In an embodiment, the present invention provides an Artificial Intelligence based hair and scalp diagnosis method. The method comprises capturing data related to hair and scalp of a patient using a plurality of TOT devices and storing the captured data in a database, identifying a characteristic information associated with the captured data and determining identifiers associated with the captured data using an AI engine, and processing the characteristic information, the identifiers, at least one data model and AI based processing logic to identify and analyze condition of the hair and scalp of the patient to recommend a treatment to the user.

In another embodiment the present invention provides a non-transitory computer readable storage medium having computer readable instructions tangibly embodied thereon which, when implemented, cause at least one computing device to carry out steps of the method of the present invention.

### BRIEF DESCRIPTION OF THE DRAWINGS

The above and still further features and advantages of embodiments of the present invention will become apparent upon consideration of the following detailed description of embodiments thereof, especially when taken in conjunction with the accompanying drawings, and wherein:

FIG. 1 shows a perspective view of hair and scalp diagnosis and treatment system in accordance with an embodiment of the present invention.

FIG. 1a shows a perspective view of hair and scalp diagnosis and treatment system with plurality of scanning devices in accordance with an embodiment of the present invention.

FIG. 1b shows a perspective view of the scanning device of the system in accordance with an embodiment of the present invention.

FIG. 1c shows a back-side view of the scanning device of the system in accordance with an embodiment of the present invention.

FIG. 1d shows an exploded horizontal view of the scanning device of the system in accordance with an embodiment of the present invention.

FIG. 1e shows an exploded vertical view of the scanning device of the system in accordance with an embodiment of the present invention.

FIG. 2 shows a flow diagram depicting a method for hair and scalp diagnosis and treatment in accordance with an embodiment of the present invention.

To facilitate understanding, like reference numerals have been used, where possible, to designate like elements common to the figures.

### DETAILED DESCRIPTION OF THE PREFERRED EMBODIMENTS

Various embodiment of the present invention provides a system and method for hair and scalp diagnosis and treatment. The following description provides specific details of certain embodiments of the invention illustrated in the drawings to provide a thorough understanding of those embodiments. It should be recognized, however, that the present invention can be reflected in additional embodiments and the invention may be practiced without some of the details in the following description.

The various embodiments including the example embodiments will now be described more fully with reference to the accompanying drawings, in which the various embodiments of the invention are shown. The invention may, however, be embodied in different forms and should not be construed as limited to the embodiments set forth herein. Rather, these embodiments are provided so that this disclosure will be thorough and complete, and will fully convey the scope of the invention to those skilled in the art. In the drawings, the sizes of components may be exaggerated for clarity.

It will be understood that when an element or layer is referred to as being “on,” “connected to,” or “coupled to” another element or layer, it can be directly on, connected to, or coupled to the other element or layer or intervening elements or layers that may be present. As used herein, the term “and/or” includes any and all combinations of one or more of the associated listed items.

Spatially relative terms, such as “image processing,” “data,” and the like, may be used herein for ease of description to describe one element or feature's relationship to another element(s) or feature(s) as illustrated in the figures. It will be understood that the spatially relative terms are intended to encompass different orientations of the structure in use or operation in addition to the orientation depicted in the figures.

Embodiments described herein will refer to plan views and/or cross-sectional views by way of ideal schematic views. Accordingly, the views may be modified depending on simplistic assembling or manufacturing technologies and/or tolerances. Therefore, example embodiments are not limited to those shown in the views but include modifications in configurations formed on basis of assembling process. Therefore, regions exemplified in the figures have schematic properties and shapes of regions shown in the figures exemplify specific shapes or regions of elements, and do not limit the various embodiments including the example embodiments.

The subject matter of example embodiments, as disclosed herein, is described with specificity to meet statutory requirements. However, the description itself is not intended to limit the scope of this patent. Rather, the inventors have contemplated that the claimed subject matter might also be embodied in other ways, to include different features or combinations of features similar to the ones described in this document, in conjunction with other technologies. Generally, the various embodiments including the example embodiments relate to system and method for diagnosis and treatment of hair and scalp.

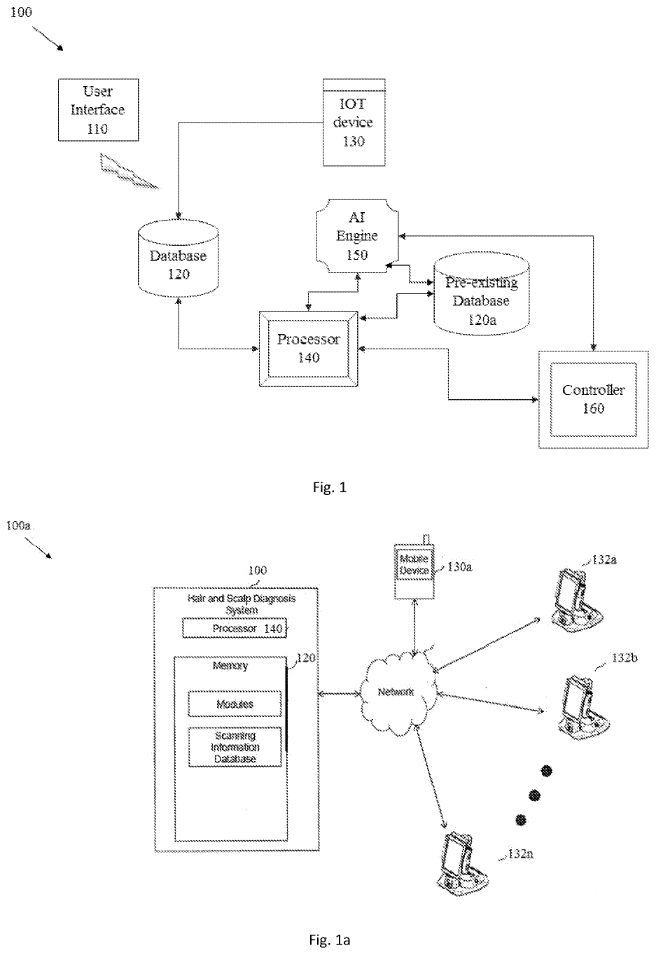

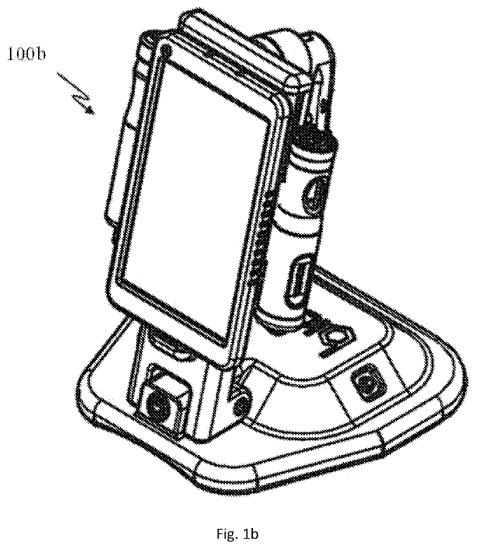

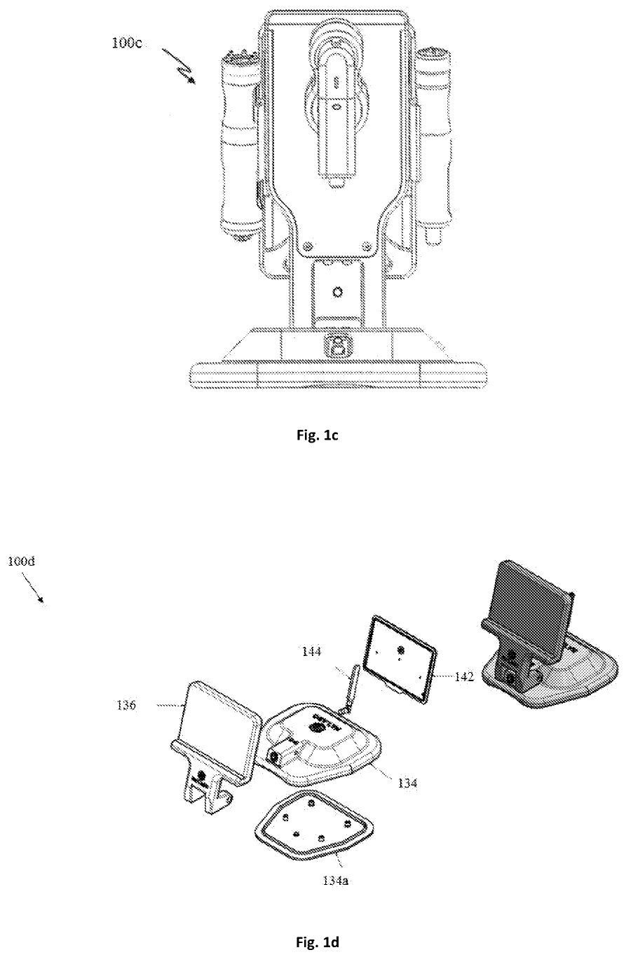

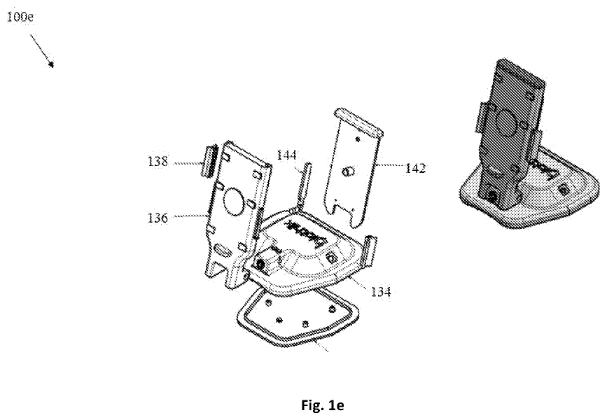

Referring to FIG. 1, an Artificial intelligence-based hair and scalp diagnosis system 100 is shown in accordance with an embodiment of the present invention. The system 100 includes an electronic user interface 110, a database 120 configured to collect hair and scalp related data from a plurality of IOT devices 130, a processor 140 coupled to an AI engine 150 configured for identifying a characteristic information associated with the captured data and determining identifiers associated with the captured data, and a controller 160 encoded with instructions to process a plurality of data models wherein the AI engine 150 determines identifiers in the data based on at least one of the plurality of data models for analyzing condition of hair and scalp to recommend the treatment to a user on the interface 110.

In an embodiment, the data is an image data or text data or a combination of both.

Referring to FIG. 1a, a diagnosis system with TOT devices 130 including mobile device 130a and plurality of scanning devices 132 (132a, 132b, . . . 132n) is shown in accordance with an embodiment of the present invention. The devices 132 are configured for scanning the image data. The scanning devices 132 scan the image data using at least two magnification levels in multiple stages and processes the image data to determine problems with the hair and scalp of the user.

In an embodiment, the identifiers are location coordinates on the image data.

In an embodiment, pixel of the image, the location coordinates of the identifiers, the at least one data model stored in a model database and AI based processing logic enables determination of the condition and recommendation of the treatment.

The AI engine 150 enables comparison of the data with a pre-existing diagnosis database 120a for identifying a closest match to determine condition of the hair and scalp (FIG. 1). Further, the AI engine 150 utilizes image processing, natural language processing and dynamically changing data model for diagnosis and recommendation of the treatment. The AI engine 150 is configured to compare the data with a standard library stored in a functional database. Also, the AI engine 150 is configured to utilize a questionnaire answered by a user.

In an embodiment, the processor 140 is configured to produce a score and rating for the data.

In an embodiment, the AI engine 150 is configured to utilize previous results, score and rating, data matrix, and a plurality of pre-defined problem types.

In an embodiment, the characteristic information includes white spots on hair or scalp, buldge on the scalp, less moisture content, dandruff etc. determined by an image processing apparatus using natural language processing, neural networks and a prediction algorithm.

In an exemplary embodiment, the processor 140 is configured to process the collected images by the database 120. In an embodiment, the processor is configured to compare the collected images with a standard library stored in the database 120. Further, in an embodiment, the processor is configured to use an artificial intelligence engine 150 to process the images. The images are processed as per a number of parameters in combination or by themselves. In an embodiment, the parameters that may be determined from head and scalp images are hair loss type, hair density, hair thickness, scalp status, balding status, and pore status. The hair loss type may be classified as M shape, or O shape or any other popular shapes. The hair density may be defined as number of hairs in area of 1 cm². This may be obtained by capturing the head and scalp image with least 60× camera lens. The hair thickness may be defined as width of a hair strand in one pore. In an example, this may be obtained by capturing head and scalp image with at least 150× camera lens. Furthermore, the scalp status can be classified as oily, dry, sensitive, dandruff and any other category.

In an embodiment, the processor 140 may also utilize a questionnaire answered by the user/customer/patient. For example, an artificial intelligence system can use natural language processing and an inference engine to analyse and understand information received in the questionnaire. Further, the artificial intelligence system may use machine learning and deep learning (such as neural network) to make predictions and recommendation for treatment, based on the processing of data. Those skilled in the art will appreciate that the machine learning will improve in efficacy over time based on training by more data and feeding back of accepted recommendation by the customer.

Further, the processor 140 may utilize the machine learning to compare the data with standard library, and analyses the various parameters as described above i.e. hair loss type, scalp status, pore status, hair thickness and hair density, and produce a score and rating for the collected image. Those skilled in the art will appreciate that the score and rating review may be fed back into the training model. Further, the processor may utilize the previous results, the score and rating, the answer matrix of questionnaire, the parameters as determined by the processor and a plurality of pre-defined problem types in recommendation of solution and product for the patient/customer. Further, the processor 140 is configured to recommend treatment for the patient's hair and scalp using the artificial intelligence. Further, the processor 140 is configured to match the score and rating with a predetermined series of solutions and products. Further, those skilled in the art will appreciate that artificial intelligence may provide new recommendation also for the hair and scalp problem of customers.

In an embodiment, the processor 140 of the present invention is implemented in the form of one or more processors and may be implemented as appropriate in hardware, computer-executable instructions, firmware, or combinations thereof. The one or more processors may reside within the hair and scalp diagnosis system. Computer-executable instruction or firmware implementations of the processing module may include computer-executable or machine-executable instructions written in any suitable programming language to perform the various functions described.

In an embodiment, the system 100 of the present invention includes memory devices that may include a permanent memory such as hard disk drive, may be configured to store data, and executable program instructions that are implemented by the processor 140. The memory devices may be implemented in the form of a primary and a secondary memory. The memory devices may store additional data and program instructions that are loadable and executable on the processor 140, as well as data generated during the execution of these programs. Further, the memory devices may be a volatile memory, such as a random-access memory and/or a disk drive, or a non-volatile memory. The memory devices may comprise of removable memory such as a Compact Flash card, Memory Stick, Smart Media, Multimedia Card, Secure Digital memory, or any other memory storage that exists currently or may exist in the future.

In an embodiment, the database 140 may include information corresponding to various scalp and hair types. The information may be in the form of images which are tagged appropriately to be uniquely identified. The database 140 may include images corresponding to plurality of scalp types, hair types, scalp conditions, hair conditions, bald pattern, and other hair and scalp diseases, among other type of information.

In an embodiment, the database 140 may include plurality of treatment that may be recommended based on the patients report. Additionally, the database 140 may also include one or more products information, which may be used for different treatments.

In an embodiment the system 100 provides an electronic user interface 110 for devices such as a touch screen device, an ultrasonic device, and scanner among other input-output devices.

In an embodiment the system 100 includes a display device configured to display content and the instruct on the scanning and recommendation process. The display device may also be used to receive an input from a user/operator. The display device may be of any display type known in the art, for example, Liquid Crystal Displays (LCD), Light Emitting Diode (LED) Displays, Cathode Ray Tube (CRT) Displays, Orthogonal Liquid Crystal Displays (OLCD) or any other type of display currently existing or which may exist in the future. The display device may be used to display the scanner output, closest results at each stage of the diagnosis process, recommended treatment information, product information among other information.

In an embodiment the system 100 of the present invention includes at least one communication device as IOT device 130 configured to provide an interface between the hair and scalp diagnosis system and other remote networks (not indicated in the diagram). The communication device may include a modem, a network interface card (such as Ethernet card), a communication port, and a Personal Computer Memory Card International Association (PCMCIA) slot, among others. The communication device may include devices supporting both wired and wireless protocols. Data in the form of electronic, electromagnetic, optical, among other signals may be transferred via the communication device to remote networks or servers.

Further, the network environment includes the hair and scalp diagnosis system connected to various scalp scanning devices via a network. The Network may include, but is not restricted to, a communication network such as Internet, PSTN, Local Area Network (LAN), Wide Area Network (WAN), Metropolitan Area Network (MAN), and so forth. In an embodiment, the network (106) can be a data network such as the Internet. Further, the messages exchanged between the hair and scalp diagnosis system and the scalp scanning devices can comprise any suitable message format and protocol capable of communicating the information necessary for the hair and scalp diagnosis system to utilize artificial intelligence and recommendation for the treatment for the customer hair and scalp problem

Referring to FIGS. 1b, 1c, 1d and 1e a perspective view 100b, a back-side view 100c, an exploded horizontal view 100d and an exploded vertical view 100e of the scanning device is shown in accordance with an embodiment of the present invention. The scanner or scanning device of the system includes a magnified lens and a camera module to scan the scalp and hair condition. The scanner may have interchangeable lens modules that may be irremovably attached to the scanner. Different stages of scanning process may require different levels of magnifying lens. Preferably, the scanner module may use a 60λ, 150× and 200× lens. The scanning device also includes base plate 134, stand 136, holder assembly 138, base plate bottom cover 134a, stand back plate 142 and Antenna 144.

In an embodiment, the scalp scanning devices are positioned in different saloons located at different locations. The scalp scanning device includes a screen, a scanner, an ultrasonic wave generator, and a jet. The scanning device is configured to scan a sample (for example, hairs and scalp of customer) from each zone using a first lens (60 times zoom lens). The scanning device is further configured to generate a scanning report of the scalp type, condition, hair type, hair loss, bald pattern, hair density, and thickness.

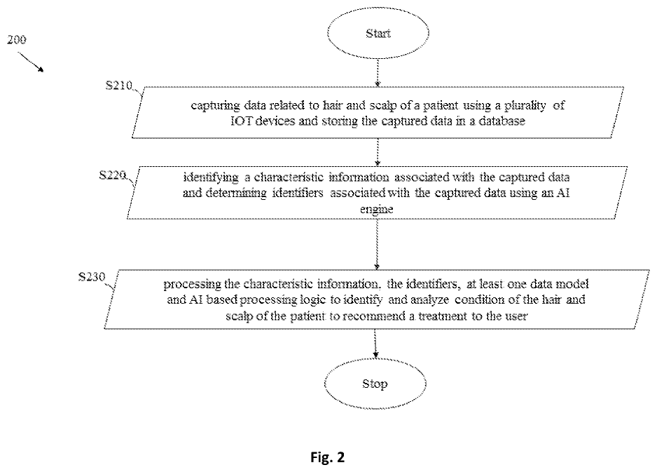

Referring to FIG. 2 a flowchart 200 depicting an Artificial Intelligence based hair and scalp diagnosis method is shown in accordance with an embodiment of the present invention. The method includes the step of S210 capturing data related to hair and scalp of a patient using a plurality of IOT devices and storing the captured data in a database, S220 identifying a characteristic information associated with the captured data and determining identifiers associated with the captured data using an AI engine, and S230 processing the characteristic information, the identifiers, at least one data model and AI based processing logic to identify and analyze condition of the hair and scalp of the patient to recommend a treatment to the user.

In an exemplary embodiment, the system and method of the present invention is implemented, provided and performed in a cloud or cloud based computing environment. In a cloud computing environment, the system is configured to save customer scanning information received from a scalp scanning device to the database. According to an embodiment of the present invention, the scalp scanning devices are positioned in different saloons located at different locations within a city or outside the city and connected through internet of things (IOT). In an embodiment, a customer with a mobile device and having authentication means (for example, QR code, mobile app, and card) may be able to gain access to the scanning device. Once the customer is registered with the scalp scanning device, the customer scanning information from the scalp scanning device may be automatically stored in the database. In an embodiment, the customer scanning information may be accessed by another scanning device upon the customer identification (for example, QR code). Those skilled in the art will appreciate that saving of customer scanning information in an online database facilitates reusing of the scanning information by other scalp scanning device, in case the customer visits different saloon.

In an embodiment the diagnosis method or process is initiated by scanning the head of the patient using a normal lens without any magnification as a first step from the top view. Further, the hair and scalp diagnosis system may divide the head image into multiple zones, preferably 5 zones i.e. front zone, right zone, left zone, back zone and middle zone. Further, the magnifier of the scanner module 106 is changed to first zoom level, preferably 60λ, to determine the scalp type, scalp and hair condition, hair type, hair loss, bald pattern, hair density, and/or hair thickness, and scalp dryness, among other information pertaining to hair and scalp. For each of such parameters the hair and scalp diagnosis system may display the one or more closest images from the database, to help the operator identify the closest match for the patient's condition. Further, the above process is repeated for all the zones to identify the appropriate scalp and hair metrics, by averaging out the results obtained from each zone.

The magnifying lens of the scanner module may be changed again to a second level, preferably to 150×, in order to determine the root condition, among other parameters. The closes images from the database may be retried by the hair and scalp diagnosis device in order to assist the operator to identify the closest match. Further, the process is repeated for all the zones and the results are averaged out to accurately determine the root condition and other required parameters.

In each of the steps, the operator is required to select the perfect match from the images automatically retrieved by the hair and scalp diagnosis system. Alternatively, the hair and scalp diagnosis system may be configured to automatically determine the perfect match at each stage using one or more image processing algorithms. Further, based on the selections at each stage and the averaged results from all the zones the hair and scalp diagnosis system may generate a diagnosis report. Finally, based on the generated report the hair and scalp diagnosis system may recommend a treatment along with appropriate products to be used by the patient in order to improve his/her hair and scalp.

In an embodiment, the pixel of the image, the location coordinates of the identifiers, the at least one data model stored in a model database and AI based processing logic enables determination of the condition and recommendation of the treatment.

In an advantageous aspect, the hair and scalp diagnosis system and the method performed by the hair and scalp diagnosis system advantageously utilizes artificial intelligence and recommends various new treatments/solutions for the hair and scalp problems of the patients/users/customer. Hence, no professional support is required in the diagnosis of hair and scalp of the patients/customers. Further, the hair and scalp diagnosis system provide storing customer's hair and scalp scanning information in a database hosted on a cloud. Hence, if customer visits any other saloon in the future, the scalp scanning device located in that saloon may access the previous scanning information of the customer stored in the database. Further, the hair and scalp diagnosis system advantageously provide recommendation for new treatment and products for the customer hair and scalp problem, without any professional involvement, thereby reducing cost of healthcare.

As would be apparent to a person having ordinary skilled in the art, the afore-described methods and components may be provided in many variations, modifications or alternatives to existing methods and systems. The principles and concepts disclosed herein may also be implemented in various manner which may not have been specifically described herein but which are to be understood as encompassed within the scope of the appended claims.

1. An Artificial intelligence-based hair and scalp diagnosis system 100, comprising
an electronic user interface 110;
a database 120 configured to collect hair and scalp related data from a plurality of IOT devices 130;
a processor 140 coupled to an AI engine 150 configured for identifying a characteristic information associated with the captured data and determining identifiers associated with the captured data; and
a controller 160 encoded with instructions to process a plurality of data models wherein the AI engine 150 determines identifiers in the data based on at least one of the plurality of data models for analyzing condition of hair and scalp to recommend the treatment to a user on the interface.

2. The system of claim 1 wherein, the data is an image data or text data or a combination of both.

3. The system of claim 2 wherein the TOT devices 130 include scanning devices configured for scanning the image data.

4. The system of claim 3 wherein the scanning devices scan the image data using at least two magnification levels in multiple stages and processes the image data to determine problems with the hair and scalp of the user.

5. The system of claim 2 wherein the identifiers are location coordinates on the image data.

6. The system of claim 5 wherein pixel of the image, the location coordinates of the identifiers, the at least one data model stored in a model database and AI based processing logic enables determination of the condition and recommendation of the treatment.

7. The system of claim 6 wherein the AI engine 150 enables comparison of the data with a pre-existing diagnosis database for identifying a closest match to determine condition of the hair and scalp.

8. The system of claim 1 wherein the system is provided in a cloud or cloud based computing environment.

9. The system of claim 5 wherein the AI engine 150 utilizes image processing, natural language processing and dynamically changing reference data models for diagnosis and recommendation of the treatment.

10. The system of claim 1, wherein the AI engine 150 is configured to compare the data with a standard library stored in a functional database.

11. The system of claim 1, wherein the AI engine 150 is configured to utilize a questionnaire answered by a user.

12. The system of claim 1, wherein the processor 140 is configured to produce a score and rating for the data.

13. The system of claim 12, wherein the AI engine 150 is configured to utilize previous results, score and rating, data matrix, and a plurality of pre-defined problem types.

14. The method of claim 9 wherein the characteristic information includes white spots on hair or scalp, buldge on the scalp, less moisture content, dandruff etc. determined by an image processing apparatus using natural language processing, neural networks and a prediction algorithm.

15. An Artificial Intelligence based hair and scalp diagnosis method, comprises:
capturing data related to hair and scalp of a patient using a plurality of IOT devices and storing the captured data in a database;
identifying a characteristic information associated with the captured data and determining identifiers associated with the captured data using an AI engine, and
processing the characteristic information, the identifiers, at least one data model and AI based processing logic to identify and analyze condition of the hair and scalp of the patient to recommend a treatment to the user.

16. The method of claim 14 wherein the method is performed in a cloud or cloud based computing environment.

17. The method of claim 14 wherein the data is an image data or text data or a combination of both.

18. The method of claim 16 wherein the TOT devices include scanning devices configured for scanning the image data.

19. The method of claim 17 wherein the scanning devices scan the image data using at least two magnification levels in multiple stages and processes the image data to determine problems with the hair and scalp of the user.

20. The method of claim 16 wherein the identifiers are location coordinates on the image data.

21. The method of claim 17 wherein pixel of the image, the location coordinates of the identifiers, the at least one data model stored in a model database and AI based processing logic enables determination of the condition and recommendation of the treatment.

22. The method of claim 14 wherein the AI engine enables comparison of the data with a pre-existing diagnosis database for identifying a closest match to determine condition of the hair and scalp.

23. The method of claim 14 wherein the AI engine utilizes image processing, natural language processing and dynamically changing the at least one data model for diagnosis and recommendation of the treatment.

24. The method of claim 23 wherein the characteristic information includes white spots, buldge on the scalp, less moisture content, dandruff etc. determined by an image processing apparatus using natural language processing, neural networks and a prediction algorithm.

25. A non-transitory computer readable storage medium having computer readable instructions tangibly embodied thereon which, when implemented, cause at least one computing device to carry out steps comprising:
capturing data related to hair and scalp of a patient using a plurality of TOT devices and storing the captured data in a database;
identifying a characteristic information associated with the captured data and determining identifiers associated with the captured data using an AI engine, and
processing the characteristic information, the identifiers, at least one data model and AI based processing logic to identify and analyze condition of the hair and scalp of the patient to recommend a treatment to the user.
